(19) United States
(12) Patent Application Publication
(10) Pub. No.: US 2014/0274758 A1
(43) Pub. Date: Sep. 18, 2014

(54) ANTIBODY PROFILING, METHODS AND APPARATUS FOR IDENTIFYING AN INDIVIDUAL OR SOURCE OF A BIOLOGICAL MATERIAL

(71) Applicant: Battelle Energy Alliance, LLC, Idaho Falls, ID (US)

(72) Inventors: William A. Apel, Jackson, WY (US); Vicki S. Thompson, Idaho Falls, ID (US); Jeffrey A. Lacey, Idaho Falls, ID (US); Shawna Park, Atlanta, GA (US)

(21) Appl. No.: 13/832,406

(22) Filed: Mar. 15, 2013

(51) Int. Cl. G01N 33/543

(52) U.S. Cl. CPC G01N 33/54306
USPC 506/9; 506/18

(57) ABSTRACT

A sample of a biological material having individual-specific antibodies is contacted with an array of less than about 200 proteins on a support to bind some of the individual-specific antibodies to the proteins of the array to form immune complexes. A detection agent with an interacting protein for conjugation to a marker is applied to the array to detect the immune complexes and obtain an antibody profile, which is compared to a known antibody profile obtained from an individual. The array may further include control spots including human IgG to form control complexes and volume assessment spots including volume determination proteins to form volume complexes. Intensity of the control complexes may be detected to determine if results of the identifying are complete. Intensity of the volume complexes may be detected to determine if a volume of the sample is sufficient for an accurate result.

### GOVERNMENT RIGHTS

This invention was made with government support under Contract Number DE-AC07-05ID14517 awarded by the United States Department of Energy. The government has certain rights in the invention.

### CROSS-REFERENCE TO RELATED APPLICATIONS

This application is related to U.S. patent application Ser. No. (Docket No. 63077.8050) for COMPUTING SYSTEMS, COMPUTER-READABLE MEDIA AND METHODS OF ANTIBODY PROFILING, filed concurrently herewith, U.S. patent application Ser. No. 12/883,002, filed Sep. 15, 2010, for “IDENTIFICATION OF DISCRIMINANT PROTEINS THROUGH ANTIBODY PROFILING, METHODS AND APPARATUS FOR IDENTIFYING AN INDIVIDUAL,” and U.S. patent application Ser. No. 12/586,109, filed Sep. 17, 2009, for “IDENTIFICATION OF DISCRIMINANT PROTEINS THROUGH ANTIBODY PROFILING, METHODS AND APPARATUS FOR IDENTIFYING AN INDIVIDUAL,” the entire contents for each of which are incorporated herein by this reference.

### STATEMENT REGARDING SEQUENCE LISTING

This application makes reference to a sequence listing that was submitted with U.S. Nonprovisional application Ser. No. 12/586,109 filed on Mar. 17, 2011. An electronic version of the Sequence Listing has been submitted with that application and is hereby incorporated by reference. Under 37 CFR 1.821(e), no new sequence listing is required to be submitted at this time, since the sequences referenced in this application are the exact same sequences referenced in Ser. No. 12/586,109 and no additional sequences are listed in this application.

### FIELD

Embodiments of the present disclosure relate to analyzing biological samples to identify proteins useful in identifying individuals, and more particularly, to methods and an apparatus for identifying an individual using such proteins.

### BACKGROUND

The importance of differentiating and identifying individuals based on biological samples with a high degree of efficiency and accuracy is presented in various contexts. For example, the need for accurate methods of identification is of increasing importance in law enforcement as it may be critical to link an individual to a forensic sample, such as blood, tissue, hair, saliva, or the like.

### SUMMARY

A method for identifying a source of a biological material that includes contacting a sample of a biological material having individual-specific antibodies with an array including multiple proteins comprising less than about 200 proteins on a support to bind at least a portion of the individual-specific antibodies to the multiple proteins of the array, to form immune complexes; applying to the array at least one detection agent that includes at least one interacting protein conjugated to a marker, and contacting the detection agent with a plurality of control spots in the array to form control complexes, wherein each control spot of the plurality includes human Immunoglobulin G; removing non-immobilized individual-specific antibodies and unbound detection agent; detecting the immune complexes on the array to obtain an antibody profile; detecting an intensity of the control complexes to determine if results of the identifying are complete; and comparing the antibody profile to a known antibody profile obtained from an individual.

A method for identifying a source of a biological material that includes contacting a sample of a biological material having individual-specific antibodies with an array including multiple proteins comprising less than about 200 proteins on a support to bind at least a portion of the individual-specific antibodies to the multiple proteins of the array, to form immune complexes; contacting the sample with a plurality of volume assessment spots in the array to form volume complexes, each volume assessment spot including a predetermined concentration of one or more volume determination proteins; applying to the array at least one detection agent comprising at least one interacting protein conjugated to a marker to detect the immune complexes and the volume complexes; removing non-immobilized individual-specific antibodies and unbound detection agent; detecting the immune complexes on the array to obtain an antibody profile; detecting an intensity of the volume complexes to determine if a volume of the sample is sufficient for an accurate result; and comparing the antibody profile to a known antibody profile obtained from an individual.

A protein array, for identifying an individual, that includes an array of multiple proteins having less than about 200 proteins immobilized on a support, wherein each protein is known, each protein is immobilized at a known predetermined location on the support, and the multiple proteins are configured to bind to at least a portion of individual-specific antibodies to form immune complexes; and a plurality of control spots as part of the array, wherein each control spot includes human Immunoglobulin G configured to form control complexes.

A protein array, for identifying an individual that includes an array of multiple proteins having less than about 200 proteins immobilized on a support, wherein each protein is known, each protein is immobilized at a known predetermined location on the support, and the multiple proteins are configured to bind to at least a portion of individual-specific antibodies to form immune complexes; and a plurality of volume assessment spots as part of the array, wherein each volume assessment spot includes a predetermined concentration of one or more volume determination proteins configured to bind to antibodies of a human to form volume complexes.

A protein array, for identifying an individual, that includes a plurality of sub-arrays, each sub-array each having an array of multiple proteins having less than about 200 proteins immobilized on a support, wherein each protein is known, each protein is immobilized at a known predetermined location on the support, and the multiple proteins are configured to bind to at least a portion of individual-specific antibodies to form immune complexes; and a plurality of control spots as part of the array, wherein each control spot includes human Immunoglobulin G configured to form control complexes.

A protein array, for identifying an individual that includes a plurality of sub-arrays, each sub-array has an array of multiple proteins that has less than about 200 proteins immobilized on a support, wherein each protein is known, each protein is immobilized at a known predetermined location on the support, and the multiple proteins are configured to bind to at least a portion of individual-specific antibodies to form immune complexes; and a plurality of volume assessment spots as part of the array, wherein each volume assessment spot includes a predetermined concentration of one or more volume determination proteins configured to bind to antibodies of a human to form volume complexes.

While the invention is susceptible to various modifications and implementation in alternative forms, specific embodiments have been shown by way of non-limiting example in the drawings and have been described in detail herein. However, it should be understood that the disclosure is not intended to be limited to the particular forms disclosed. Rather, the disclosure includes all modifications, equivalents, and alternatives falling within the scope of the disclosure as defined by the following appended claims and their legal equivalents.

### BRIEF DESCRIPTION OF THE DRAWINGS

While the specification concludes with claims particularly pointing out and distinctly claiming that which is regarded as the present invention, advantages of this disclosure may be more readily ascertained from the following description of the disclosure when read in conjunction with the accompanying drawings in which:

FIG. 1 shows a protein array according to an embodiment of the present disclosure;

FIG. 2 shows a protein array including control spots and volume assessment spots according to one or more embodiments of the present disclosure; and

FIG. 3 shows a super array including three protein arrays according to one or more embodiments of the present disclosure.

### DETAILED DESCRIPTION

Before embodiments of the present disclosure are described in detail, it is to be understood that this disclosure is not limited to the particular configurations, process acts, and materials disclosed herein as such configurations, process acts, and materials may vary somewhat. It is also to be understood that the terminology employed herein is used for the purpose of describing particular embodiments only and is not limiting since the scope of the present disclosure will be limited only by the appended claims and equivalents thereof.

The publications and other reference materials referred to herein to describe the background of the disclosure and to provide additional detail regarding its practice. The references discussed herein are provided solely for their disclosure prior to the filing date of the present application. Nothing herein is to be construed as an admission that such documents constitute prior art, or that the inventors are not entitled to antedate such disclosure by virtue of prior invention.

While the known methods for using antibody profiling are generally suitable for their limited purposes, they possess certain inherent deficiencies that detract from their overall utility in analyzing, characterizing, and identifying biological samples. For example, the known methods rely on fractionation of antigens by electrophoresis and then transfer of the fractionated antigens to a membrane. Due to differences in conditions from one fractionation procedure to another, there are lot-to-lot differences in the positions of the antigens on the membrane such that results obtained using membranes from one lot cannot be compared with results obtained using membranes from another lot. Further, when colorimetric procedures are used for detecting immune complexes on the membrane, color determination may be subjective such that results may be interpreted differently by different observers.

It would be advantageous to provide a method identifying proteins capable of distinguishing an individual and methods for efficiently and accurately determining identity, distinguishing between individuals, as well as determining the source of biological fluids, especially those amenable to automation.

It must be noted that, as used in this specification and the appended claims, the singular forms “a,” “an,” and “the” include plural referents unless the context clearly dictates otherwise. Thus, for example, reference to a method for analyzing a biological sample from “an animal” includes reference to two or more of such animals, reference to “a support” includes reference to one or more of such supports, and reference to “an array” includes reference to two or more of such arrays.

As used herein, “blood” means and includes whole blood, plasma, serum, or any derivative of blood. A blood sample may be, for example, serum.

As used herein, “comprising,” “including,” “containing,” “characterized by,” and grammatical equivalents thereof are inclusive or open-ended terms that do not exclude additional, unrecited elements or method acts. “Comprising” is to be interpreted as including the more restrictive terms “consisting of” and “consisting essentially of”

As used herein, “consisting of” and grammatical equivalents thereof exclude any element, step, or ingredient not specified in the claim.

As used herein, “consisting essentially of” and grammatical equivalents thereof limit the scope of a claim to the specified materials or acts and those that do not materially affect the basic and novel characteristic or characteristics of the claimed invention.

As used herein, the terms “biological sample” and “sample” mean and include a sample comprising individual-specific antibodies obtained from an organism or from components (e.g., cells) of an organism. The sample may be of any biological material. Such samples include, but are not limited to, blood, blood fractions (e.g., serum, plasma), blood cells (e.g., white cells), tissue or fine needle biopsy samples, urine, saliva, perspiration or semen. Biological samples may also include sections of tissues such as frozen sections taken for histological purposes.

As used herein, “color marker” refers to a substrate that produces a colored product in the visible light spectrum upon digestion with an appropriate enzyme. Such colored markers are distinguished from digestion that my produce fluorescent and luminescent products.

The term “discriminant analysis” means and includes a set of statistical methods used to select features that optimally discriminate between two or more groups. Application of discriminant analysis to a data set allows the user to focus on the most discriminating features for further analysis.

As used herein, the terms “immobilized” or “affixed” mean and include an association between a protein or antigen and a substrate at the molecular level (i.e., through a covalent or non-covalent bond or interaction). For example, a protein may be immobilized to a support by covalent bonding directly to a surface of the support which may or may not be modified to enhance such covalent bonding. Also, the protein may be immobilized to the support by use of a linker molecule between the protein and the support. Proteins may further be immobilized on the support by steric hindrance within a polymerized gel or by covalent bonding within a polymerized gel. Proteins may also be immobilized on a support through hybridization between the protein and a molecule immobilized on the support.

The term “protein array” as used herein refers to a protein array, a protein macroarray, a protein microarray or a protein nanoarray. A protein array may include, for example, but is not limited to, ProtoArray™ high density protein array, which is commercially available from Invitrogen (Carlsbad, Calif.). The ProtoArray™ high density protein array may be used to screen complex biological mixtures, such as serum, to assay for the presence of autoantibodies directed against human proteins. Alternatively, a custom protein array that includes autoantigens, such as those provided herein, for the detection of autoantibody biomarkers, may be used to assay for the presence of autoantibodies directed against human proteins. In certain disease states including autoimmune diseases and cancer, autoantibodies are expressed at altered levels relative to those observed in healthy individuals.

As used herein, “support” means a generally or substantially planar substrate onto which an array of antigens is disposed. A support may comprise any material or combination of materials suitable for carrying the array. Materials used to construct these supports need to meet several requirements, such as (1) the presence of surface groups that may be easily derivatized, (2) inertness to reagents used in the assay, (3) stability over time, and (4) compatibility with biological samples. For example, suitable materials include glass, silicon, silicon dioxide (i.e., silica), plastics, polymers, hydrophilic inorganic supports, and ceramic materials. Illustrative plastics and polymers include poly(tetrafluoroethylene), poly(vinylidenedifluoride), polystyrene, polycarbonate, polymethacrylate, and combinations thereof. Illustrative hydrophilic inorganic supports include alumina, zirconia, titania, and nickel oxide. An example of a glass substrate would be a microscope slide. Silicon wafers used to make computer chips have also been used to make biochips. See, e.g., U.S. Pat. No. 5,605,662. The supports may further include a coating, such as, nitrocellulose, gelatin, a polymer (i.e., polyvinyl difluoride) or an aldehyde.

As used herein, a “complex” refers to the binding of one molecule to another through a non-covalent interaction, such as the binding of an antibody to an antigen.

In some embodiments, a method of determining proteins useful in discriminating one individual from 1 or more other individuals and/or positively identifying an individual is provided. Such proteins may be referred to herein as “discriminant proteins.” The method may employ a protein array including a plurality of proteins immobilized on a support. As a non-limiting example, the protein array may be a ProtoArray™ human protein microarray, which is commercially available from Invitrogen Corporation (Carlsbad, Calif.). The plurality of proteins immobilized on the support may include a plurality of antigens.

In a typical assay, a plurality of biological samples including individual-specific antibodies may each be physically contacted with a protein array, under conditions that permit high affinity binding, but that minimize non-specific interactions. In one embodiment, the biological samples are introduced to the protein array that includes a plurality of antigens immobilized in predetermined locations on a support. The protein array may be washed free of unbound material, and the presence of bound antibodies may be detected, and correlated with the cognate antigen.

The data collected from each of the plurality of biological samples profiled on a protein array may be used to determine an antibody profile for the individual. The antibody profiles may be analyzed using, for example, conventional discriminant analysis methods, to determine proteins relevant in discriminating and positively identifying an individual (i.e., discriminant proteins) from a population of one or more other individuals. The discriminant proteins may be used to generate a test panel for identifying an individual or determining a source of a biological sample. In some embodiments, the test panel may be, for example, a protein array 100, as shown in FIG. 1, including a plurality of the discriminant proteins arranged as spots 104 in predetermined locations on a support 102.

### Protein Array

The protein array may be prepared by attaching the antigens to the surface of the support 102 in a preselected pattern such that the locations of antigens in the array are known. As used herein, an antigen is a substance that is bound by an antibody. Antigens may include proteins, carbohydrates, nucleic acids, hormones, drugs, receptors, tumor markers, and the like, and mixtures thereof. An antigen may also be a group of antigens, such as a particular fraction of proteins eluted from a size exclusion chromatography column. Still further, an antigen may also be identified as a designated clone from an expression library or a random epitope library.

In one embodiment, antigens may be isolated from HeLa cells as generally described in A. M. Francoeur et al., Identification of Ki (Ku, p70/p80) Autoantigens and Analysis of Anti-Ki Autoantibody Reactivity, 136 J. Immunol. 1648 (1986). Briefly, HeLa cells may be grown in standard medium under standard tissue culture conditions. Confluent HeLa cell cultures may then be rinsed, preferably with phosphate-buffered saline (PBS), lysed with detergent, and centrifuged to remove insoluble cellular debris. The supernate contains approximately 10,000 immunologically distinct antigens suitable for generating an array.

There is no requirement that the antigens used to generate the array be known. All that is required is that the source of the antigens be consistent such that a reproducible array may be generated. For example, the HeLa cell supernate containing the antigens may be fractionated on a size exclusion column, electrophoretic gel, density gradient, or the like, as is well known in the art. Fractions may be collected, and each fraction collected could represent a unique set of antigens for the purpose of generating the array. Thus, even though the antigens may be unknown, a reproducible array may be generated if the HeLa cell antigens may be isolated and fractionated using the same method and conditions.

Other methods, such as preparation of random peptide libraries or epitope libraries are well known in the art and may be used to reproducibly produce antigens (e.g., J. K. Scott and G. P. Smith, Searching for Peptide Ligands with an Epitope Library, 249 Science 386 (1990); J. J. Devlin et al., Random Peptide Libraries: A Source of Specific Protein Binding Molecules, 249 Science 404-406 (1990); S. E. Cwirla et al., Peptides on Phage: A Vast Library of Peptides for Identifying Ligands, 87 Proc. Nat'l Acad. Sci. USA 6378-6382 (1990); K. S. Lam et al., A New Type of Synthetic Peptide Library for Identifying Ligand-binding Activity, 354 Nature 82-84 (1991); S. Cabilly, Combinatorial Peptide Library Protocols, Humana Press, 304 p.p., 129-154 1997; and U.S. Pat. No. 5,885,780). Such libraries may be constructed by ligating synthetic oligonucleotides into an appropriate fusion phage. Fusion phages may be filamentous bacteriophage vectors in which foreign sequences may be cloned into phage gene III and displayed as part of the gene III protein (pIII) at one tip of the virion. Each phage encodes a single random sequence and expresses it as a fusion complex with pIII, a minor coat protein present at about five molecules per phage. For example, in the fusion phage techniques of J. K. Scott and G. P. Smith, supra, a library was constructed of phage containing a variable cassette of six amino acid residues. The hexapeptide modules fused to bacteriophage proteins provided a library for the screening methodology that may examine >10¹² phages (or about 10⁸-10¹⁰ different clones) at one time, each with a test sequence on the virion surface. The library obtained was used to screen monoclonal antibodies specific for particular hexapeptide sequences. The fusion phage system has also been used by other groups, and libraries containing longer peptide inserts have been constructed. Fusion phage prepared according to this methodology may be selected randomly or non-randomly for inclusion in the array of antigens. The fusion phages selected for inclusion in the array may be propagated by standard methods to result in what is virtually an endless supply of the selected antigens.

Other methods for producing antigens are also known in the art. For example, expression libraries may be prepared by random cloning of DNA fragments or cDNA into an expression vector (e.g., R. A. Young and R. W. Davis, Yeast RNA Polymerase II Genes: Isolation with Antibody Probes, 222 Science 778-782 (1983); G. M. Santangelo et al., Cloning of Open Reading Frames and Promoters from the Saccharomyces cerevisiae Genome: Construction of Genomic Libraries of Random Small Fragments, 46 Gene 181-186 (1986). Expression vectors that could be used for making such libraries are commercially available from a variety of sources. For example, random fragments of HeLa cell DNA or cDNA may be cloned into an expression vector, and then clones expressing HeLa cell proteins may be selected. These clones may then be propagated by methods well known in the art. The expressed proteins may then be isolated or purified and may be used in the making of the array.

Alternatively, antigens may be synthesized using recombinant DNA technology well known in the art. Genes that code for many proteins from a gamut of organisms including viruses, bacteria, and mammals have been cloned, and thus large quantities of highly pure proteins may be synthesized quickly and inexpensively. For example, the genes that code for many eukaryotic and mammalian membrane-bound receptors, growth factors, cell adhesion molecules, and regulatory proteins have been cloned and may be useful as antigens. Many proteins produced by such recombinant techniques, such as transforming growth factor, acidic and basic fibroblast growth factors, interferon, insulin-like growth factor, and various interleukins from different species, are commercially available. In most instances, the entire polypeptide need not be used as an antigen. For example, any size or portion of the polypeptide that contains at least one epitope, i.e., antigenic determinant or portion of an antigen that specifically interacts with an antibody, will suffice for use in the array. In addition, a particular antigen may be purified or isolated from any natural or synthetic source of the antigen by methods known in the art.

The antigens, whether selected randomly or non-randomly, may be disposed on the support to result in the array. The pattern of the antigens on the support should be reproducible. In embodiments, the location and identity of each antigen on the support may be known. For example, in a 10×10 array one skilled in the art might place antigens 1-100 in locations 1-100, respectively, of the array. As a non-limiting example, each of the antigens of the array may be deposited on the support 102 as a spot 104 having a diameter of from about 10 microns to about 500 microns and, more particularly, from about 50 microns to about 300 microns.

The proteins may placed in arrays on the surface of the support 102 using a pipetting device or a machine or device configured for placing liquid samples on the support 102, for example, using a commercially available microarrayer, such as those from Arrayit Corporation (Sunnyvale, Calif.); Genomic Solutions, Inc. (Ann Arbor, Mich.); Gene Machines (San Carlos, Calif.); Genetic MicroSystems, Inc. (Woburn, Mass.); GenePack DNA (Cambridge, UK); Genetix Ltd. (Christchurch, Dorset, UK); and Packard Instrument Company (Meriden, Conn.).

Relevant methods to array a series of proteins onto a surface include contact printing processes, non-contact printing processes and in silico protein synthesis arrayer processes. Commercially available instruments are available for both methods. In some embodiments, conventional contact printing processes, such as contact pin printing and microstamping, in which the printing device may physically contact a surface may be used to apply the proteins to the surface of the support 102. For example, a pin printing device such as that commercially available from Arrayit Corporation may be used to deposit spots 104 having an average diameter of 65 microns or larger. As another non-limiting example, Genomic Solutions offers several nanoliter dispensing instruments that may dispense liquid volumes from 20 nL up to 250 μL from 96-, 384-, 1536-, 3456-, and 9600-well microtiter plates and place them precisely on a surface with densities up to 400 spots/cm². The instruments will spot onto surfaces in a variety of patterns. In additional embodiments, the protein antigens may be applied to the surface without physical contact between the printing device and the surface using conventional non-contact printing processes including, but not limited to, photochemistry-based methods, laser writing, electrospray deposition, and inkjet. As the name implies, inkjet technology utilizes the same principles as those used in inkjet printers. MicroFab Technologies, Inc. (Plano, Tex.), offers a ten-fluid print head that may dispense picoliter quantities of liquids onto a surface in a variety of patterns. An illustrative pattern for the present application would be a simple array ranging from 10×10 up to 100×100. The protein antigens may be applied to the surface using a serial deposition process or a parallel deposition process.

There are a number of methods that may be used to attach proteins or other antigens to the surface of the support 102. The simplest of these is simple adsorption through hydrophobic, ionic, and van der Waals forces. As a non-limiting example, bifunctional organosilanes may be used in attachment of proteins to the surface of the support (e.g., Thompson and Maragos, Fiber-Optic Immunosensor for the Detection of Fumonisin B₁, 44 J. Agric. Food Chem. 1041-1046 (1996)). One end of the organosilane reacts with exposed —OH groups on the surface of the support to form a silanol bond. The other end of the organosilane contains a group that is reactive with various groups on the protein surface, such as —NH₂ and —SH groups. This method of attaching proteins to the support results in the formation of a covalent linkage between the protein and the support. Other suitable methods that have been used for protein attachment to surfaces include arylazide, nitrobenzyl, and diazirine photochemistry methodologies. Exposure of the above chemicals to UV light causes the formation of reactive groups that may react with proteins to form a covalent bond. The arylazide chemistry forms a reactive nitrene group that may insert into C—H bonds, while the diazirine chemistry results in a reactive carbene group. The nitrobenzyl chemistry is referred to as caging chemistry whereby the caging group inactivates a reactive molecule. Exposure to UV light frees the molecule and makes it available for reaction. Still other methods for attaching proteins to supports are well known in the art, (e.g., S. S. Wong, Chemistry of Protein Conjugation and Cross-Linking CRC Press, 340, 1991).

Following attachment of the antigens on the support 102 in the selected array, the support 102 may be washed. The wash solution may include, for example, one or more of a surfactant or a non-specific protein such as bovine serum albumin (BSA). Appropriate liquids for washing include, but are not limited to, phosphate buffered saline (PBS) and the like, i.e., relatively low ionic strength, biocompatible salt solutions buffered at or near neutrality. Many of such appropriate wash liquids are known in the art or may be devised by a person skilled in the art without undue experimentation (e.g., N. E. Good and S. Izawa, Hydrogen Ion Buffers, 24 Methods Enzymology 53-68 (1972)).

The support 102 may be processed for blocking of nonspecific binding of proteins and other molecules to the support. This blocking step may prevent the binding of antigens, antibodies, and the like to the support wherein such antigens, antibodies, or other molecules are not intended to bind. Blocking may reduce the background that might swamp out the signal, thus increasing the signal-to-noise ratio. The support 102 may be blocked by incubating the support 102 in a medium that contains inert molecules that bind to sites where nonspecific binding might otherwise occur. Examples of suitable blockers include, but are not limited to, bovine serum albumin, human albumin, gelatin, nonfat dry milk, polyvinyl alcohol, TWEEN® 20, and various commercial blocking buffers, such as SEABLOCK™ blocking buffer from EastCoast Bio, Inc., (West Berwick, Me.) and SUPERBLOCK® blocking buffer from Pierce Chemical Co., (Rockford, Ill.). In some embodiments, one or more of the suitable blockers may be incorporated into the wash solution described above.

### Antibody Profile

The array may be contacted with a sample of the biological material to be tested. For example, the biological sample may be obtained from various bodily fluids and solids, including blood, saliva, semen, serum, plasma, urine, amniotic fluid, pleural fluid, cerebrospinal fluid, and mixtures thereof. These biological samples may be obtained according to methods well known in the art. Depending on the detection method used, it may be required to manipulate the biological sample to attain optimal reaction conditions. For example, the ionic strength or hydrogen ion concentration or the concentration of the biological sample may be adjusted for optimal immune complex formation, enzymatic catalysis, and the like.

Antibodies (immunoglobulins) are a family of variable glycoproteins that bind specifically to foreign molecules (antigens). The binding strength between an antigen (epitope) and antigen-binding site in an antibody (paratope) is termed affinity. Each antibody has a minimum of two antigen-binding sites, and is thus multivalent to its antigen. The strength of a single antigen-antibody bond is termed the antibody affinity and it is produced by the number of bonds between the antigen and the antibody. The binding strength is greatly increased with more bonds because all of the antigen-antibody bonds must be broken simultaneously before the antigen and antibody can dissociate. Even when each antigen-binding site has a low affinity, antibodies can function effectively.

Antibodies have a variable region (FAB fragment) and a constant region (FC region) and both regions have antigen-binding sites that can be used for detection.

As described in detail in U.S. Pat. No. 5,270,167 to Francoeur, when ISAs are allowed to react with a set of random antigens, a certain number of immune complexes form. For example, using a panel of about 1000 unique antigens, about 30 immune complexes between ISAs in a biological sample that has been diluted 20-fold may be detected. If the biological sample is undiluted, the total number of possible detectable immune complexes that could form would be greater than 10²³. The total number of possible immune complexes may also be increased by selecting “larger” antigens, i.e., proteins instead of peptides) that have multiple epitopes. Therefore, it will be appreciated that depending on the antigens and number thereof used, the dilution of the biological sample, and the detection method, one skilled in the art may regulate the number of immune complexes that will form and be detected. As used herein, an “antibody profile” refers to the set of unique immune complexes that form and fail to form between the ISAs in the biological sample and the antigens in the array.

Detection and/or Quantification of Reactions

Methods for detecting antibody/antigen or immune complexes are well known in the art. The present disclosure may be modified by one skilled in the art to accommodate the various detection methods known in the art. The particular detection method chosen by one skilled in the art depends on several factors, including the amount of biological sample available, the type of biological sample, the stability of the biological sample, the stability of the antigen, and the affinity between the antibody and antigen. Moreover, as discussed above, depending on the detection methods chosen, it may be required to modify the biological sample. While these techniques are well known in the art, non-limiting examples of a few of the detection methods that may be used to practice embodiments of the present disclosure are briefly described below.

There are many types of immunoassays known in the art. The most common types of immunoassay are competitive and non-competitive heterogeneous assays, such as, for example, enzyme-linked immunosorbent assays (ELISAs). In a non-competitive ELISA, unlabeled antigen is bound to a support. A biological sample may be combined with antigens bound to the reaction vessel, and antibodies (primary antibodies) in the biological sample may be allowed to bind to the antigens, forming the immune complexes. After the immune complexes have formed, excess biological sample may be removed and the array may be washed to remove nonspecifically bound antibodies. The immune complexes may then be reacted with an appropriate enzyme-labeled anti-immunoglobulin (secondary antibody). The secondary antibody reacts with antibodies in the immune complexes, not with other antigens bound to the array. Secondary antibodies specific for binding antibodies of different species, including humans, are well known in the art and are commercially available, such as from Sigma Chemical Co. (St. Louis, Mo.) and Santa Cruz Biotechnology, Inc. (Santa Cruz, Calif.). After an optional further wash, the enzyme substrate may be added. The enzyme linked to the secondary antibody catalyzes a reaction that converts the substrate into a product. When excess antigen is present, the amount of product is directly proportional to the amount of primary antibody present in the biological sample. By way of non-limiting example, the product may be fluorescent or luminescent, which may be measured using technology and equipment well known in the art. It is also possible to use reaction schemes that result in a colored product, which may be measured spectrophotometrically.

In other embodiments of the disclosure, the secondary antibody may not be labeled to facilitate detection. Additional antibodies may be layered (i.e., tertiary, quaternary, etc.) such that each additional antibody specifically recognizes the antibody previously added to the immune complex. Any one of these additional (i.e., tertiary, quaternary, etc.) may be labeled so as to allow detection of the immune complex as described herein.

Sandwich or capture assays may also be used to identify and quantify immune complexes. Sandwich assays are a mirror image of non-competitive ELISAs in that antibodies are bound to the solid phase and antigen in the biological sample is measured. These assays may be particularly useful in detecting antigens having multiple epitopes that are present at low concentrations. This technique requires excess antibody to be attached to a solid phase. The bound antibody is then incubated with the biological samples, and the antigens in the sample may be allowed to form immune complexes with the bound antibody. The immune complex is incubated with an enzyme-linked secondary antibody, which recognizes the same or a different epitope on the antigen as the primary antibody. Hence, enzyme activity is directly proportional to the amount of antigen in the biological sample. D. M. Kemeny and S. J. Challacombe, ELISA and Other Solid Phase Immunoassays, (John Wiley & Sons Ltd.) (1988).

Typical enzymes that may be linked to secondary antibodies include, but are not limited to, horseradish peroxidase, glucose oxidase, glucose-6-phosphate dehydrogenase, alkaline phosphatase, β-galactosidase, and urease. Secondary antigen-specific antibodies linked to various enzymes are commercially available from, for example, Sigma Chemical Co. and Amersham Life Sciences (Arlington Heights, Ill.).

Competitive ELISAs are similar to noncompetitive ELISAs except that enzyme linked antibodies compete with unlabeled antibodies in the biological sample for limited antigen binding sites. Briefly, a limited number of antigens may be bound to the support. Biological sample and enzyme-labeled antibodies may be added to the support 102. Antigen-specific antibodies in the biological sample compete with enzyme-labeled antibodies for the limited number of antigens bound to the support 102. After immune complexes have formed, nonspecifically bound antibodies may be removed by washing, enzyme substrate is added, and the enzyme activity is measured. No secondary antibody is required. Because the assay is competitive, enzyme activity is inversely proportional to the amount of antibodies in the biological sample.

Another competitive ELISA may also be used within the scope of the present disclosure. In this embodiment, limited amounts of antibodies from the biological sample may be bound to the surface of the support as described herein. Labeled and unlabeled antigens may be then brought into contact with the support such that the labeled and unlabeled antigens compete with each other for binding to the antibodies on the surface of the support. After immune complexes have formed, nonspecifically bound antigens may be removed by washing. The immune complexes may be detected by incubation with an enzyme-linked secondary antibody, which recognizes the same or a different epitope on the antigen as the primary antibody, as described above. The activity of the enzyme is then assayed, which yields a signal that is inversely proportional to the amount of antigen present. Homogeneous immunoassays may also be used when practicing the method of the present disclosure. Homogeneous immunoassays may be preferred for detection of low molecular weight compounds, such as hormones, therapeutic drugs, and illegal drugs that cannot be analyzed by other methods, or compounds found in high concentration. Homogeneous assays may be particularly useful because no separation step is necessary. R. C. Boguslaski et al., Clinical Immunochemistry: Principles of Methods and Applications, (1984).

In homogeneous techniques, bound or unbound antigens may be enzyme-linked. When antibodies in the biological sample bind to the enzyme-linked antigen, steric hindrances inactivate the enzyme. This results in a measurable loss in enzyme activity. Free antigens (i.e., not enzyme-linked) compete with the enzyme-linked antigen for limited antibody binding sites. Thus, enzyme activity is directly proportional to the concentration of antigen in the biological sample.

Enzymes useful in homogeneous immunoassays include, but are not limited to, lysozyme, neuraminidase, trypsin, papain, bromelain, glucose-6-phosphate dehydrogenase, and β-galactosidase. T. Persoon, “Immunochemical Assays in the Clinical Laboratory,” 5 Clinical Laboratory Science 31 (1992). Enzyme-linked antigens are commercially available or may be linked using various chemicals well known in the art, including glutaraldehyde and maleimide derivatives.

Prior antibody profiling technology involved an alkaline phosphatase labeled secondary antibody with 5-bromo-4-chloro-3′-indolylphosphate p-toluidine salt (BCIP) and nitro-blue tetrazolium chloride (NBT), both of which are commercially available from a variety of sources, such as from Pierce Chemical Co. (Rockford, Ill.). The enzymatic reaction forms an insoluble colored product that is deposited on the surface of membrane strips to form bands wherever antigen-antibody complexes occur. As a non-limiting example, the array may be scanned to detect a colored product using one of a variety of conventional desktop scanners, which are commercially available from a variety of sources, such as from Canon U.S.A. (Lake Success, N.Y.). The intensity of the colored product may be quantified by calculating the median feature pixel intensity minus median background pixel intensity.

As another non-limiting example, gold nanoparticle labeled antibodies may be employed and may be detected using a scanning, transmission electron microscopy, and/or dark-field zoom stereomicroscopy. Compared to conventional fluorescent labels, the gold nanoparticles scatter incident white light to generate monochromatic light which may be easily detected. The light intensity generated by the gold nanoparticles may be up to 100,000 times greater than that generated by fluorescent-labeled molecules. For example, the gold nanoparticles may be detected using a conventional desktop scanner. Han et al., Detection of Analyte Binding to Microarrays Using Gold Nanoparticle Labels and a Desktop Scanner, 3 Lab Chip 329; 329-332 (2003).

Fluorescent immunoassays may also be used when practicing the method of the present disclosure. Fluorescent immunoassays are similar to ELISAs except the enzyme is substituted for fluorescent compounds called fluorophores or fluorochromes. These compounds have the ability to absorb energy from incident light and emit the energy as light of a longer wavelength and lower energy. Fluorescein and rhodamine, usually in the form of isothiocyanates that may be readily coupled to antigens and antibodies, are most commonly used in the art. D. P. Stites et al., Basic and Clinical Immunology, (1994). Fluorescein absorbs light of 490 to 495 nm in wavelength and emits light at 520 nm in wavelength. Tetramethylrhodamine absorbs light of 550 nm in wavelength and emits light at 580 nm in wavelength. Illustrative fluorescence-based detection methods include ELF-97 alkaline phosphatase substrate (Molecular Probes, Inc., Eugene, Oreg.); PBXL-1 and PBXL-3 (phycobilisomes conjugated to streptavidin) (Martek Biosciences Corp., Columbia, Md.); FITC (fluorescein isothiocyanate) and Texas Red labeled goat anti-human IgG (Jackson ImmunoResearch Laboratories, Inc., West Grove, Pa.); and B-Phycoerythrin and R-Phycoerythrin conjugated to streptavidin (Molecular Probes Inc.). ELF-97 is a nonfluorescent chemical that is digested by alkaline phosphatase to form a fluorescent molecule. Because of turnover of the alkaline phosphatase, use of the ELF-97 substrate results in signal amplification. Fluorescent molecules attached to secondary antibodies do not exhibit this amplification.

Phycobiliproteins isolated from algae, porphyrins, and chlorophylls, which all fluoresce at about 600 nm, are also being used in the art. I. Hemmila, Fluoroimmunoassays and Immunofluorometric Assays, 31 Clin. Chem. 359 (1985); U.S. Pat. No. 4,542,104. Phycobiliproteins and derivatives thereof are commercially available under the names R-phycoerythrin (PE) and QUANTUM RED™ from Sigma Chemical Co.

In addition, Cy-conjugated secondary antibodies and antigens may be useful in immunoassays and are commercially available. Cy3, for example, is maximally excited at 554 nm and emits light at between 568 and 574 nm. Cy3 is more hydrophilic than other fluorophores and thus has less of a tendency to bind nonspecifically or aggregate. Cy-conjugated compounds are commercially available from Amersham Life Sciences.

Illustrative luminescence-based detection methods include CSPD® and CDP star alkaline phosphatase substrates from Roche Molecular Biochemicals, (Indianapolis, Ind.) and SUPERSIGNAL® horseradish peroxidase substrate from Pierce Chemical Co., (Rockford, Ill.).

Chemiluminescence, electroluminescence, and electrochemiluminescence (ECL) detection methods may also be attractive means for quantifying antigens and antibodies in a biological sample. Luminescent compounds have the ability to absorb energy, which is released in the form of visible light upon excitation. In chemiluminescence, the excitation source is a chemical reaction; in electroluminescence the excitation source is an electric field; and in ECL an electric field induces a luminescent chemical reaction.

Molecules used with ECL detection methods generally comprise an organic ligand and a transition metal. The organic ligand forms a chelate with one or more transition metal atoms forming an organometallic complex. Various organometallic and transition metal-organic ligand complexes have been used as ECL labels for detecting and quantifying analytes in biological samples. Due to their thermal, chemical, and photochemical stability, their intense emissions and long emission lifetimes, ruthenium, osmium, rhenium, iridium, and rhodium transition metals are favored in the art. The types of organic ligands are numerous and include anthracene and polypyridyl molecules and heterocyclic organic compounds. For example, bipyridyl, bipyrazyl, terpyridyl, and phenanthrolyl, and derivatives thereof, are common organic ligands in the art. A common organometallic complex used in the art includes tris-bipyridine ruthenium (II), commercially available from IGEN, Inc. (Rockville, Md.) and Sigma Chemical Co.

ECL may be performed under aqueous conditions and under physiological pH, thus minimizing biological sample handling. J. K. Leland et al., Electrogenerated Chemiluminescence: An Oxidative-Reduction Type ECL Reactions Sequence Using Triprophyl Amine, 137 J. Electrochemical Soc. 3127-3131 (1990); WO 90/05296; and U.S. Pat. No. 5,541,113. Moreover, the luminescence of these compounds may be enhanced by the addition of various cofactors, such as amines.

A tris-bipyridine ruthenium (II) complex, for example, may be attached to a secondary antibody using strategies well known in the art, including attachment to lysine amino groups, cysteine sulfhydryl groups, and histidine imidazole groups. In a typical ELISA immunoassay, secondary antibodies would recognize antibodies bound to antigens, but not unbound antigens. After washing nonspecific binding complexes, the tris-bipyridine ruthenium (II) complex may be excited by chemical, photochemical, and electrochemical excitation means, such as by applying current to the array (e.g., WO 86/02734). The excitation would result in a double oxidation reaction of the tris-bipyridine ruthenium (II) complex, resulting in luminescence that could be detected by, for example, a photomultiplier tube. Instruments for detecting luminescence are well known in the art and are commercially available, for example, from IGEN, Inc. (Rockville, Md.).

Solid state color detection circuitry may also be used to monitor the color reactions on the array and, on command, compare the color patterns before and after the sample application. A color camera image may also be used and the pixel information analyzed to obtain the same information.

Still another method involves detection using a surface plasmon resonance (SPR) chip. The surface of the chip is scanned before and after sample application and a comparison is made. The SPR chip relies on the refraction of light when the molecules of interest may be exposed to a light source. Each molecule has its own refraction index by which it may be identified. This method requires precise positioning and control circuitry to scan the chip accurately.

In one embodiment, the detecting agents bind to specific portions of the antibodies. Antibodies have a variable region (FAB fragment) and a constant region (FC region) and both regions have antigen-binding sites that can be used for detection. By capitalizing on both regions of the antibody through multiple combinations, the array can essentially determine the quantity of antibodies present in the biological sample and the structural integrity (quality) of these antibodies by measuring the intensity from each of the bound spots.

Chosen antibodies can be conjugated to human IgG so that they can be detected directly in vitro by the anti-Human detection conjugate (AHG). AHG is used to detect in vitro (array) sensitization and detection of anti-red cell antibodies in serum or plasma. These monoclonal antibodies recognize an exposed surface determinant of intact red blood cells and will bind to the Fc (constant region) receptors. In addition, other monoclonal antibodies specific for an Fc portion of human IgG can be used on the array which recognizes an epitope common to all human IgG subclasses or can be specific for a FAB portion of human IgG, which in turn would be non-reactive with the Fc portion of human IgG, eliminating antigen-binding affinity competition. Polyclonal antibodies specific for the Fc or FAB portion may also be used, which is specific to human IgG only and will not bind to other Igs (immunoglobulins).

Utilizing all or a combination of the described antibodies to determine the quantity of antibodies present in the sample and the structural integrity of these antibodies, the colorigenic marker can directly detect the proportion of bound sample to serve as sample control at the time of assay. This eliminates the use of unnecessary sample tests as well as ensuring or discrediting the generated antibody profile in real time.

Yet another method involves a fluid rinse of the array with a fluorescing reagent. The antigens that combine with the biological sample will fluoresce and may be detected with a charge-coupled device (CCD) array. The output of such a CCD array is analyzed to determine the unique pattern associated with each sample. Speed is not a factor with any of the methods since the chemical combining of sample and reference takes minutes to occur.

Moreover, array scanners are commercially available, such as from Genetic MicroSystems, Inc. The GMS 418 Array Scanner uses laser optics to rapidly move a focused beam of light over the array. This system uses a dual-wavelength system including high-powered, solid-state lasers that generate high excitation energy to allow for reduced excitation time. At a scanning speed of 30 Hz, the GMS 418 may scan a 22×75-mm slide with 10-μm resolution in about four minutes.

Software for image analysis obtained with an array scanner is readily available. Available software packages include ImaGene (BioDiscovery, Los Angeles, Calif.); ScanAlyze (available at no charge; developed by Mike Eisen, Stanford University, Palo Alto, Calif.); De-Array (developed by Yidong Chen and Jeff Trent of the National Institutes of Health; used with IP Lab from Scanalytics, Inc., Fairfax, Va.); Pathways (Research Genetics, Huntsville, Ala.); GEM Tools (Incyte Pharmaceuticals, Inc., Palo Alto, Calif.); and Imaging Research (Amersham Pharmacia Biotech, Inc., Piscataway, N.J.).

Once interactions between the antigens and antibodies have been identified and quantified, the signals may be digitized. The digitized antibody profile may serve as a signature that identifies the source of the biological sample. Depending on the array used, the digitized data may take numerous forms. For example, the array may include 10 columns and 10 rows for a total number of 100 spots, each including at least one antigen. After the biological sample including the antibodies is added to the array and allowed to incubate, interactions between antigens and antibodies in the biological sample may be identified and quantified. In each spot, an interaction between the antigen in the spot and the antibody in the biological sample will either result in or not result in a quantifiable signal. In one embodiment, the results of the antibody profile may be digitized by, by way of non limiting example, ascribing each one of the 100 spots a numerical value of either “0,” if a quantifiable signal was not obtained, or “1,” if a quantifiable signal was obtained. Using this method, the digitized antibody profile may comprise a unique set of zeroes and ones. It will be understood that the use of 1 and 0 is merely exemplary and that any set of values or indicators may be used to signify the absence, presence, or intensity of a particular signal.

The numerical values “0” or “1” may, of course, be normalized to signals obtained in internal control spots so that digitized antibody profiles obtained at a later time may be properly compared. For example, one or several of the spots may contain a known antigen, which will remain constant over time. Therefore, if a subsequent biological sample is more or less dilute than a previous biological sample, the signals may be normalized using the signals from the known antigen.

It will be appreciated by one skilled in the art that other methods of digitizing the antibody profile exist and may be used. For example, rather than ascribing each spot with a numerical value of “0” or “1,” the numerical value may be incremental and directly proportional to the strength of the signal.

### Statistical Analysis

The antibody profiles obtained from the plurality of individuals may be analyzed using conventional discriminant analysis methods to determine proteins useful in discriminating or identifying an individual from one or more other individuals. For example, discriminant proteins may be determined using forward selection, backward elimination, or stepwise selection to determine a subset of proteins that best reveals differences among the classes (i.e., the individuals). The STEPDISC procedure, which is available from SAS Institute, Inc. (Cary, N.C.), may be used to perform a stepwise discriminant analysis to select a subset of the proteins useful in discriminating among individuals. Signals from a set of proteins that make up each class may be assumed to be multivariate normal with a common covariance matrix.

Using the STEPDISC procedure, variables (in particular, signals from particular proteins) may be chosen to enter or leave the model according to the significance level of an F-test from an analysis of covariance, where the variables already chosen act as covariates and the variable under consideration is the dependent variable. In other embodiments, a variable could be chosen to enter or leave the model according to whether the squared partial correlation for its prediction using the class variable (and controlling for the effects of the other variables already in the model) is high.

In some embodiments, the discriminant proteins useful in discriminating or identifying an individual may be determined by calculating various discriminant functions for classifying observations using the protein signals. Linear or quadratic discriminant functions may be used for data with approximately multivariate normal within-class distributions. Nonparametric methods may be used without making any assumptions about these distributions.

One or more of the discriminant proteins may be used to identify an individual, to distinguish between individuals, or to establish or rule out the source of a biological sample. In some embodiments, one or more of the discriminant proteins may be used as part of a test panel. For example, discriminant proteins may be immobilized on a support in the form of an array as described above to form a protein array useful in discriminating among individuals and/or sources of a biological sample. However, other methods of detecting an interaction between a discriminant protein and an antibody present in a biological sample, such as conventional protein affinity chromatography methods, affinity blotting methods, immunoprecipitation methods, and cross-linking methods, may also be used. In embodiments, the array or test panel may be used to generate an antibody profile which may be used to distinguish between individuals in a population, or to establish or rule out the source of a biological sample within a population, wherein the population may comprise 1 million, 10 million, 100 million, 1 billion, 10 billion, 100 billion, or more individuals.

The array may include several discriminant proteins, each of which may be immobilized on a support. The array may include less than about 200, 175, 170, 150, 125, 110, 100, 75, or 50 discriminant proteins. For example, the test panel for discriminating or identifying an individual may include from about 20 to about 90 discriminant proteins, and more particularly, from about 45 to about 80 discriminant proteins, less than about 100 discriminant proteins, less than about 110 discriminant proteins, or less than about 170 discriminant proteins. With “X” different profiles that are each independent, the probability that no two different people have the same profile among “m” people can be shown to be equal to exp[−m*m/(2×)]. As a non-limiting example, greater than about 76 independent discriminant proteins may be used to distinguish an individual among a population of about 10 billion individuals, the probability of a match between two different individuals being less than about 0.0001. As another non-limiting example, greater than about 86 independent discriminant proteins may be used to distinguish an individual among a population of about 100 billion individuals, the probability of a match between two different individuals being less than about 0.0001. Examples of discriminant proteins include, but are not limited to, those proteins presented in Table 1.

In another embodiment, an array has sub-arrays and each sub-array may include less than about 200, 175, 170, 150, 125, 110, 100, 75, or 50 discriminant proteins. For example, each sub-array for the test panel for discriminating or identifying an individual may include from about 20 to about 90 discriminant proteins, and more particularly, from about 45 to about 80 discriminant proteins, less than about 100 discriminant proteins, less than about 110 discriminant proteins, or less than about 170 discriminant proteins. Comparing the detected immune complexes between each sub-array leads to greater confidence in identification.

TABLE 1

| SEQ ID NO | Protein ID |
|---|---|
| SEQ ID NO: 1 | PM_2149 |
| SEQ ID NO: 2 | PM_2151 |
| SEQ ID NO: 3 | BC010125.1 |
| SEQ ID NO: 4 | BC011414.1 |
| SEQ ID NO: 5 | BC012945.1 |
| SEQ ID NO: 6 | BC014409.1 |
| SEQ ID NO: 7 | BC015219.1 |
| SEQ ID NO: 8 | BC016470.2 |
| SEQ ID NO: 9 | BC018206.1 |
| SEQ ID NO: 10 | BC018404.1 |
| SEQ ID NO: 11 | BC019039.2 |
| SEQ ID NO: 12 | BC019315.1 |
| SEQ ID NO: 13 | BC021189.2 |
| SEQ ID NO: 14 | BC023152.1 |
| SEQ ID NO: 15 | BC026175.1 |
| SEQ ID NO: 16 | BC026346.1 |
| SEQ ID NO: 17 | BC032825.2 |
| SEQ ID NO: 18 | BC033711.1 |
| SEQ ID NO: 19 | BC036123.1 |
| SEQ ID NO: 20 | BC040949.1 |
| SEQ ID NO: 21 | BC050377.1 |
| SEQ ID NO: 22 | BC052805.1 |
| SEQ ID NO: 23 | BC053602.1 |
| SEQ ID NO: 24 | BC060824.1 |
| SEQ ID NO: 25 | NM_015138.2 |
| SEQ ID NO: 26 | NM_175887.2 |
| SEQ ID NO: 27 | NM_000394.2 |
| SEQ ID NO: 28 | NM_000723.3 |
| SEQ ID NO: 29 | NM_001008220.1 |
| SEQ ID NO: 30 | NM_001106.2 |
| SEQ ID NO: 31 | NM_001312.2 |
| SEQ ID NO: 32 | NM_001537.1 |
| SEQ ID NO: 33 | NM_002737 |
| SEQ ID NO: 34 | NM_002740 |
| SEQ ID NO: 35 | NM_002744 |
| SEQ ID NO: 36 | NM_003907.1 |
| SEQ ID NO: 37 | NM_003910.2 |
| SEQ ID NO: 38 | NM_004064.2 |
| SEQ ID NO: 39 | NM_004394.1 |
| SEQ ID NO: 40 | NM_004845.3 |
| SEQ ID NO: 41 | NM_004965.3 |
| SEQ ID NO: 42 | NM_005030 |
| SEQ ID NO: 43 | NM_005246.1 |
| SEQ ID NO: 44 | NM_006007.1 |
| SEQ ID NO: 45 | NM_006218.2 |
| SEQ ID NO: 46 | NM_006628.4 |
| SEQ ID NO: 47 | NM_006819.1 |
| SEQ ID NO: 48 | NM_012472.1 |
| SEQ ID NO: 49 | NM_014240.1 |
| SEQ ID NO: 50 | NM_014245.1 |
| SEQ ID NO: 51 | NM_014460.2 |
| SEQ ID NO: 52 | NM_014622.4 |
| SEQ ID NO: 53 | NM_014891.1 |
| SEQ ID NO: 54 | NM_014943.3 |
| SEQ ID NO: 55 | NM_015149.2 |
| SEQ ID NO: 56 | NM_015417.2 |
| SEQ ID NO: 57 | NM_015509.2 |
| SEQ ID NO: 58 | NM_016096.1 |
| SEQ ID NO: 59 | NM_016520.1 |
| SEQ ID NO: 60 | NM_017855.2 |
| SEQ ID NO: 61 | NM_017949.1 |
| SEQ ID NO: 62 | NM_018326.1 |
| SEQ ID NO: 63 | NM_018584.4 |
| SEQ ID NO: 64 | NM_024718.2 |
| SEQ ID NO: 65 | NM_024826.1 |
| SEQ ID NO: 66 | NM_025241.1 |
| SEQ ID NO: 67 | NM_032345.1 |
| SEQ ID NO: 68 | NM_032368.3 |
| SEQ ID NO: 69 | NM_079420.1 |
| SEQ ID NO: 70 | NM_080390.3 |
| SEQ ID NO: 71 | NM_138623.2 |
| SEQ ID NO: 72 | NM_145796.2 |
| SEQ ID NO: 73 | NM_153757.1 |
| SEQ ID NO: 74 | NM_177973.1 |
| SEQ ID NO: 75 | NM_178010.1 |
| SEQ ID NO: 76 | NM_199124.1 |
| SEQ ID NO: 77 | NM_201262.1 |
| SEQ ID NO: 78 | NM_203284.1 |
| SEQ ID NO: 79 | NM_205853.1 |
| SEQ ID NO: 80 | NM_212540.1 |

In embodiments of the disclosure, a protein array may comprise 1, 2, 3, 4, 5, 10, 15, 20, 25, 50, or more discriminant proteins selected from the group consisting of SEQ ID NOs: 1-80, SEQ ID NOs: 1-45, SEQ ID NOs: 1-3, 5, 6, 8, 9, 11, 12, 15-18, 22-24, 26, 27, 29, 33, 38, 41, 44, 46-48, 51, 20, 54, 57-60, 62, 65, 68, 70, 72, 72-75, 77, and 79 and SEQ ID NOs: 1-9, 11-13, 15-20, 22-24, 26-30, 33, 35, 36, 38-41, 44, 46-54, 57-60, 62, 63, 66, 68, 70, and 72-80. In embodiments, a protein array may consist of SEQ ID NOs: 1-80, SEQ ID NOs: 1-45, SEQ ID NOs: 1-3, 5, 6, 8, 9, 11, 12, 15-18, 22-24, 26, 27, 29, 33, 38, 41, 44, 46-48, 51, 20, 54, 57-60, 62, 65, 68, 70, 72, 72-75, 77, and 79 and SEQ ID NOs: 1-9, 11-13, 15-20, 22-24, 26-30, 33, 35, 36, 38-41, 44, 46-54, 57-60, 62, 63, 66, 68, 70, and 72-80.

In embodiments of the disclosure, a protein sub-array may comprise 1, 2, 3, 4, 5, 10, 15, 20, 25, 50, or more discriminant proteins selected from the group consisting of SEQ ID NOs: 1-80, SEQ ID NOs: 1-45, SEQ ID NOs: 1-3, 5, 6, 8, 9, 11, 12, 15-18, 22-24, 26, 27, 29, 33, 38, 41, 44, 46-48, 51, 20, 54, 57-60, 62, 65, 68, 70, 72, 72-75, 77, and 79 and SEQ ID NOs: 1-9, 11-13, 15-20, 22-24, 26-30, 33, 35, 36, 38-41, 44, 46-54, 57-60, 62, 63, 66, 68, 70, and 72-80. In embodiments, a protein array may consist of SEQ ID NOs: 1-80, SEQ ID NOs: 1-45, SEQ ID NOs: 1-3, 5, 6, 8, 9, 11, 12, 15-18, 22-24, 26, 27, 29, 33, 38, 41, 44, 46-48, 51, 20, 54, 57-60, 62, 65, 68, 70, 72, 72-75, 77, and 79 and SEQ ID NOs: 1-9, 11-13, 15-20, 22-24, 26-30, 33, 35, 36, 38-41, 44, 46-54, 57-60, 62, 63, 66, 68, 70, and 72-80.

In an embodiment of the disclosure, a protein array including discriminant proteins may be used for forensic analysis for matching a biological sample to an individual such as, for example, a criminal suspect. Forensic samples obtained from crime scenes are often subject to drying of the samples, small sample sizes, mixing with samples from more than one individual, adulteration with chemicals, and the like. The present method provides the advantages of rapid analysis, simplicity, low cost, and accuracy for matching forensic samples with suspects. For example, the forensic sample and a sample from one or more suspects may be obtained according to methods well known in the art. The samples may be tested against the array and compared. If the discriminant proteins obtained from the samples match, it may be concluded that the forensic sample was obtained from the matching suspect. If no match of discriminant proteins is obtained, then none of the suspects was the source of the forensic sample.

### Example

Serum samples from ninety-four (94) individuals were profiled against a high throughput protein array with over 8000 proteins and the data from these chips was statistically analyzed to determine proteins useful for discriminating among sets of individuals in a population. The ninety-four (94) individuals included nineteen (19) Asian individuals, twenty (20) African American individuals, twenty (20) Native American individuals, and thirty-five (35) Caucasian individuals. For quality assurance (QA), the arrays contained the immobilized proteins in pairs on a support. Thus, each array provided two opportunities for antigen/antibody binding for each protein.

The serum samples were diluted 1:150 and used to probe human ProtoArray™. The arrays were blocked for 1 hour and then incubated with the serum samples for 90 minutes at about 4° C. without shaking. The arrays were then transferred to ice and washed about three times by adding about 20 ml buffer (1×PBS, 5 mM MgCl2, 0.5 mM DTT, 0.05% Triton X-100, 5% Glycerol, 1% BSA) to the arrays, incubating the arrays with the buffer for 8 minutes at 4° C., and decanting the buffer from the arrays by inverting. The arrays were incubated with anti-human IgG antibody conjugated to AlexaFluor 647 for about 90 minutes, washed as above and dried. The arrays were scanned using a ScanArray Express® 3.0 HT microarray scanner, which is available commercially from Perkin Elmer, Inc. (Waltham, Mass.). The images were captured from the microarray scanner using a 633 nm laser with the scanner set to 10 μm resolution. Following scanning, data was acquired using ImaGene 8.0 microarray analysis software from BioDiscovery (El Segundo, Calif.). Background-subtracted signals from each population were normalized utilizing a quantile normalization strategy. Subjects were distinguished from one another using conventional discriminant analysis. The STEPDISC procedure from SAS Institute, Inc. was utilized to identify discriminant proteins based on the logarithms of the intensities detected. The discriminant proteins of interest were identified as significant in distinguishing between individuals. A list of 80 discrimininating proteins from among the over 8,000 on the arrays was determined. The 80 discriminating proteins are listed in Table 2.

TABLE 2

| SEQ ID NO | Protein ID | SelOrdAll | MinPSeeOrNot | sRatio | maxCorrAfter |
|---|---|---|---|---|---|
| SEQ ID NO: 1 | PM_2149 | 16 | 0.45 | 22.1 | 0.683 |
| SEQ ID NO: 2 | PM_2151 | 99 | 0.25 | 13.4 | 0.585 |
| SEQ ID NO: 3 | BC010125.1 | 62 | 0.23 | 15.6 | 0.500 |
| SEQ ID NO: 4 | BC011414.1 | 15 | 0.40 | 19.9 | 0.482 |
| SEQ ID NO: 5 | BC012945.1 | 38 | 0.33 | 18.4 | 0.570 |
| SEQ ID NO: 6 | BC014409.1 | . | 0.32 | 10.7 | 0.448 |
| SEQ ID NO: 7 | BC015219.1 | 76 | 0.29 | 15.6 | 0.652 |
| SEQ ID NO: 8 | BC016470.2 | 74 | 0.19 | 14.6 | 0.579 |
| SEQ ID NO: 9 | BC018206.1 | 31 | 0.38 | 16.1 | 0.551 |
| SEQ ID NO: 10 | BC018404.1 | 93 | 0.27 | 19.0 | 0.754 |
| SEQ ID NO: 11 | BC019039.2 | 33 | 0.41 | 17.2 | 0.544 |
| SEQ ID NO: 12 | BC019315.1 | 27 | 0.48 | 17.8 | 0.846 |
| SEQ ID NO: 13 | BC021189.2 | 29 | 0.34 | 17.2 | 0.488 |
| SEQ ID NO: 14 | BC023152.1 | 6 | 0.10 | 25.3 | 0.752 |
| SEQ ID NO: 15 | BC026175.1 | 50 | 0.39 | 15.6 | 0.582 |
| SEQ ID NO: 16 | BC026346.1 | 78 | 0.48 | 16.4 | 0.360 |
| SEQ ID NO: 17 | BC032825.2 | 13 | 0.10 | 18.9 | 0.491 |
| SEQ ID NO: 18 | BC033711.1 | 72 | 0.29 | 14.6 | 0.567 |
| SEQ ID NO: 19 | BC036123.1 | 101 | 0.35 | 15.0 | 0.649 |
| SEQ ID NO: 20 | BC040949.1 | 45 | 0.37 | 17.9 | 0.523 |
| SEQ ID NO: 21 | BC050377.1 | 70 | 0.14 | 11.0 | 0.310 |
| SEQ ID NO: 22 | BC052805.1 | 56 | 0.29 | 16.6 | 0.501 |
| SEQ ID NO: 23 | BC053602.1 | 42 | 0.32 | 16.1 | 0.621 |
| SEQ ID NO: 24 | BC060824.1 | 12 | 0.28 | 19.4 | 0.421 |
| SEQ ID NO: 25 | NM_015138.2 | 91 | 0.33 | 13.3 | 0.607 |
| SEQ ID NO: 26 | NM_175887.2 | 34 | 0.43 | 15.4 | 0.537 |
| SEQ ID NO: 27 | NM_000394.2 | 44 | 0.38 | 20.2 | 0.737 |
| SEQ ID NO: 28 | NM_000723.3 | 200 | 0.22 | 9.4 | 0.580 |
| SEQ ID NO: 29 | NM_001008220.1 | 17 | 0.22 | 21.7 | 0.405 |
| SEQ ID NO: 30 | NM_001106.2 | 22 | 0.41 | 20.3 | 0.303 |
| SEQ ID NO: 31 | NM_001312.2 | 81 | 0.42 | 13.2 | 0.619 |
| SEQ ID NO: 32 | NM_001537.1 | 84 | 0.49 | 23.5 | 0.733 |
| SEQ ID NO: 33 | NM_002737 | 73 | 0.47 | 10.0 | 0.300 |
| SEQ ID NO: 34 | NM_002740 | 79 | 0.28 | 12.4 | 0.620 |
| SEQ ID NO: 35 | NM_002744 | 3 | 0.42 | 22.4 | 0.215 |
| SEQ ID NO: 36 | NM_003907.1 | 57 | 0.37 | 14.8 | 0.440 |
| SEQ ID NO: 37 | NM_003910.2 | 63 | 0.12 | 12.7 | 0.594 |
| SEQ ID NO: 38 | NM_004064.2 | 54 | 0.20 | 13.8 | 0.422 |
| SEQ ID NO: 39 | NM_004394.1 | 58 | 0.48 | 16.3 | 0.641 |
| SEQ ID NO: 40 | NM_004845.3 | 30 | 0.25 | 18.0 | 0.432 |
| SEQ ID NO: 41 | NM_004965.3 | 97 | 0.46 | 11.4 | 0.648 |
| SEQ ID NO: 42 | NM_005030 | 95 | 0.41 | 14.2 | 0.683 |
| SEQ ID NO: 43 | NM_005246.1 | 77 | 0.22 | 9.3 | 0.625 |
| SEQ ID NO: 44 | NM_006007.1 | 80 | 0.24 | 13.3 | 0.417 |
| SEQ ID NO: 45 | NM_006218.2 | 90 | 0.24 | 8.2 | 0.573 |
| SEQ ID NO: 46 | NM_006628.4 | 66 | 0.29 | 15.0 | 0.538 |
| SEQ ID NO: 47 | NM_006819.1 | 4 | 0.22 | 17.9 | 0.356 |
| SEQ ID NO: 48 | NM_012472.1 | 11 | 0.49 | 23.0 | 0.578 |
| SEQ ID NO: 49 | NM_014240.1 | 19 | 0.44 | 18.9 | 0.459 |
| SEQ ID NO: 50 | NM_014245.1 | 18 | 0.29 | 22.9 | 0.676 |
| SEQ ID NO: 51 | NM_014460.2 | 21 | 0.32 | 19.7 | 0.414 |
| SEQ ID NO: 52 | NM_014622.4 | 65 | 0.49 | 15.7 | 0.566 |
| SEQ ID NO: 53 | NM_014891.1 | 32 | 0.23 | 19.1 | 0.343 |
| SEQ ID NO: 54 | NM_014943.3 | 71 | 0.16 | 12.7 | 0.519 |
| SEQ ID NO: 55 | NM_015149.2 | 96 | 0.18 | 11.4 | 0.665 |
| SEQ ID NO: 56 | NM_015417.2 | 8 | 0.12 | 19.3 | 0.353 |
| SEQ ID NO: 57 | NM_015509.2 | 43 | 0.23 | 12.8 | 0.554 |
| SEQ ID NO: 58 | NM_016096.1 | 41 | 0.28 | 16.0 | 0.516 |
| SEQ ID NO: 59 | NM_016520.1 | 60 | 0.38 | 13.3 | 0.471 |
| SEQ ID NO: 60 | NM_017855.2 | 69 | 0.29 | 14.2 | 0.578 |
| SEQ ID NO: 61 | NM_017949.1 | 49 | 0.16 | 16.2 | 0.630 |
| SEQ ID NO: 62 | NM_018326.1 | 26 | 0.39 | 17.5 | 0.254 |
| SEQ ID NO: 63 | NM_018584.4 | 7 | 0.37 | 21.7 | 0.448 |
| SEQ ID NO: 64 | NM_024718.2 | 103 | 0.17 | 11.0 | 0.495 |
| SEQ ID NO: 65 | NM_024826.1 | 20 | 0.41 | 17.8 | 0.328 |
| SEQ ID NO: 66 | NM_025241.1 | 48 | 0.43 | 13.2 | 0.268 |
| SEQ ID NO: 67 | NM_032345.1 | 85 | 0.16 | 13.4 | 0.765 |
| SEQ ID NO: 68 | NM_032368.3 | 39 | 0.36 | 19.2 | 0.635 |
| SEQ ID NO: 69 | NM_079420.1 | 51 | 0.45 | 14.0 | 0.643 |
| SEQ ID NO: 70 | NM_080390.3 | 86 | 0.23 | 15.3 | 0.582 |
| SEQ ID NO: 71 | NM_138623.2 | 67 | 0.12 | 14.4 | 0.538 |
| SEQ ID NO: 72 | NM_145796.2 | 64 | 0.26 | 11.4 | 0.590 |
| SEQ ID NO: 73 | NM_153757.1 | 46 | 0.46 | 16.8 | 0.402 |
| SEQ ID NO: 74 | NM_177973.1 | 10 | 0.26 | 18.5 | 0.290 |
| SEQ ID NO: 75 | NM_178010.1 | 9 | 0.31 | 16.8 | 0.124 |
| SEQ ID NO: 76 | NM_199124.1 | 28 | 0.38 | 14.0 | 0.252 |
| SEQ ID NO: 77 | NM_201262.1 | 14 | 0.27 | 17.5 | 0.118 |
| SEQ ID NO: 78 | NM_203284.1 | 5 | 0.31 | 26.9 | 0.277 |
| SEQ ID NO: 79 | NM_205853.1 | 25 | 0.44 | 17.7 | 0.208 |
| SEQ ID NO: 80 | NM_212540.1 | 75 | 0.17 | 12.4 | . |

The discriminant proteins of Table 2 were selected to discriminate an individual based on the primary criterion that the logarithms of the associated intensity signals appear as selected variables in a STEPDISC model. Several STEPDISC models were tested. One used only data from the first QA sample associated with each protein. A second model used only data from the other QA sample. A third model used average values, and a fourth used all the data (a total of 198 sets of protein intensity data from 99 non-blank arrays). The “SelOrdAll” column in Table 1 shows the order of selection of proteins from the fourth model. The values are ranked, so “1” corresponds to the first protein selected, “2” for the second, and so forth. The protein (SEQ ID NO: 6) with no value in this column was selected in a fifth STEPDISC model that used just data from subjects with replication (specifically, data from the two individuals with more than one array in the data set were used in this model). The fourth run identified a total of 80 proteins.

The initial list was refined using three additional filters. First, proteins retained on the list had to have the between-subject standard deviation as the largest of the estimated standard deviations. The standard deviations for this filter were obtained using a conventional “components of variance” analysis for each protein that sought variation between subjects, arrays, spots on the array and the QA sampling variation. The ratio of the between-subject estimate divided by the QA sample standard deviation estimate is shown in the “sRatio” column of Table 1. This ratio was used as a further criteria in narrowing the selection (see further below).

The second criterion used in refining the list of discriminant proteins to get just 80 was related to the probability of detection. For the example embodiment of the disclosure, a median intensity of greater than 1500 was assumed to be required in order to observe the presence of antigen/antibody bonding for a protein. The fraction of array data exceeding 1500 was tabulated for each protein. In initial data screening, this fraction was required to be at least 0.1 and less than 0.9. If nearly all the sample intensities are invisible, or nearly all are visible, there is less potential for discriminating between people. The minimum of the probability of visibility, and 1—this probability, was used further as described below. This attribute of a protein is denoted as “MinPSeeOrNot” in Table 2.

To determine the subset of 45 discriminant proteins listed in Table 3 below, pairwise correlation coefficients for all pairs among the 80 proteins were evaluated. The correlations were estimated using the data set of people with just one array per person (92 arrays), so that complete independence in the results would be ideal. The correlations were estimated using JMP® statistical software from SAS Institute. For each of the 80 proteins, a maximum correlation was identified. The pair of proteins in the array with the maximum correlation of all of these was identified. The protein in this pair with other relatively high correlations was identified as the worst protein from the correlation standpoint. This protein was recorded and then all correlations associated with it were removed from further consideration. This process was repeated using the remaining data, leading to identification of the second-worst protein and its highest correlation, conditioned on the first (worst) protein being omitted. This process was repeated until only two proteins remained in the set of data being considered. These are the two most “independent” proteins among the set of 80. The maximum correlation estimated between a given protein and some other protein, given that the more highly-correlated proteins have been removed from the data set, is shown as “MaxCorrAfter” in Table 2. The most discriminating proteins have the lowest values for “MaxCorrAfter.”

The 45 discriminant proteins in Table 2 were identified using the following cutoff values for the three filters discussed above: sRatio greater than or equal to about 11, a “MaxCorrAfter” less than about 0.6, and “MinPSeeOrNot” greater than about 0.2. The numbers in this filter were selected by trial and error to retain exactly 45 proteins.

TABLE 3
45 proteins, sorted on sRatio.

| Protein ID | SEQ ID NO | selOrdAll | MinPSeeOrNot | sRatio | maxCorrAfter |
|---|---|---|---|---|---|
| NM_203284.1 | SEQ ID NO: 78 | 5 | 0.3131 | 26.9 | 0.277 |
| NM_012472.1 | SEQ ID NO: 48 | 11 | 0.4949 | 23.0 | 0.578 |
| NM_002744 | SEQ ID NO: 35 | 3 | 0.4192 | 22.4 | 0.215 |
| NM_018584.4 | SEQ ID NO: 63 | 7 | 0.3737 | 21.7 | 0.448 |
| NM_001008220.1 | SEQ ID NO: 29 | 17 | 0.2172 | 21.7 | 0.405 |
| NM_001106.2 | SEQ ID NO: 30 | 22 | 0.4091 | 20.3 | 0.303 |
| BC011414.1 | SEQ ID NO: 4 | 15 | 0.4040 | 19.9 | 0.482 |
| NM_014460.2 | SEQ ID NO: 51 | 21 | 0.3182 | 19.7 | 0.414 |
| BC060824.1 | SEQ ID NO: 24 | 12 | 0.2828 | 19.4 | 0.421 |
| NM_014891.1 | SEQ ID NO: 53 | 32 | 0.2323 | 19.1 | 0.343 |
| NM_014240.1 | SEQ ID NO: 49 | 19 | 0.4444 | 18.9 | 0.459 |
| NM_177973.1 | SEQ ID NO: 74 | 10 | 0.2576 | 18.5 | 0.290 |
| BC012945.1 | SEQ ID NO: 5 | 38 | 0.3333 | 18.4 | 0.570 |
| NM_004845.3 | SEQ ID NO: 40 | 30 | 0.2525 | 18.0 | 0.432 |
| NM_006819.1 | SEQ ID NO: 47 | 4 | 0.2222 | 17.9 | 0.356 |
| BC040949.1 | SEQ ID NO: 20 | 45 | 0.3737 | 17.9 | 0.523 |
| NM_024826.1 | SEQ ID NO: 65 | 20 | 0.4141 | 17.8 | 0.328 |
| NM_205853.1 | SEQ ID NO: 79 | 25 | 0.4394 | 17.7 | 0.208 |
| NM_018326.1 | SEQ ID NO: 62 | 26 | 0.3939 | 17.5 | 0.254 |
| NM_201262.1 | SEQ ID NO: 77 | 14 | 0.2727 | 17.5 | 0.118 |
| BC021189.2 | SEQ ID NO: 13 | 29 | 0.3434 | 17.2 | 0.488 |
| BC019039.2 | SEQ ID NO: 11 | 33 | 0.4091 | 17.2 | 0.544 |
| NM_178010.1 | SEQ ID NO: 75 | 9 | 0.3081 | 16.8 | 0.124 |
| NM_153757.1 | SEQ ID NO: 73 | 46 | 0.4596 | 16.8 | 0.402 |
| BC052805.1 | SEQ ID NO: 22 | 56 | 0.2879 | 16.6 | 0.501 |
| BC026346.1 | SEQ ID NO: 16 | 78 | 0.4798 | 16.4 | 0.360 |
| BC018206.1 | SEQ ID NO: 9 | 31 | 0.3838 | 16.1 | 0.551 |
| NM_016096.1 | SEQ ID NO: 58 | 41 | 0.2828 | 16.0 | 0.516 |
| NM_014622.4 | SEQ ID NO: 52 | 65 | 0.4899 | 15.7 | 0.566 |
| BC026175.1 | SEQ ID NO: 15 | 50 | 0.3889 | 15.6 | 0.582 |
| BC010125.1 | SEQ ID NO: 3 | 62 | 0.2323 | 15.6 | 0.500 |
| NM_175887.2 | SEQ ID NO: 26 | 34 | 0.4293 | 15.4 | 0.537 |
| NM_080390.3 | SEQ ID NO: 70 | 86 | 0.2273 | 15.3 | 0.582 |
| NM_006628.4 | SEQ ID NO: 46 | 66 | 0.2929 | 15.0 | 0.538 |
| NM_003907.1 | SEQ ID NO: 36 | 57 | 0.3737 | 14.8 | 0.440 |
| BC033711.1 | SEQ ID NO: 18 | 72 | 0.2929 | 14.6 | 0.567 |
| NM_017855.2 | SEQ ID NO: 60 | 69 | 0.2879 | 14.2 | 0.578 |
| NM_199124.1 | SEQ ID NO: 76 | 28 | 0.3788 | 14.0 | 0.252 |
| NM_004064.2 | SEQ ID NO: 38 | 54 | 0.2020 | 13.8 | 0.422 |
| PM_2151 | SEQ ID NO: 2 | 99 | 0.2475 | 13.4 | 0.585 |
| NM_016520.1 | SEQ ID NO: 59 | 60 | 0.3838 | 13.3 | 0.471 |
| NM_006007.1 | SEQ ID NO: 44 | 80 | 0.2424 | 13.3 | 0.417 |
| NM_025241.1 | SEQ ID NO: 66 | 48 | 0.4343 | 13.2 | 0.268 |
| NM_015509.2 | SEQ ID NO: 57 | 43 | 0.2273 | 12.8 | 0.554 |
| NM_145796.2 | SEQ ID NO: 72 | 64 | 0.2576 | 11.4 | 0.590 |

FIG. 2 shows a protein array 200 including control spots 210 and volume assessment spots 220 according to one or more embodiments of the present disclosure. As with the embodiment of FIG. 1, a support 202 includes a plurality of spots 204 arranged in an array. These spots 204 may include any of the proteins as described above and be arranged in any of the arrangements described above.

Control spots 210 may be included in the embodiment of FIG. 2. The control spots 210 may be used during image capture and analysis of the protein array 200 as an image registration tool to assist the image capture and analysis tools determination of where other spots 204 in the protein array 200 are relative to the control spots 210. FIG. 2 illustrates the control spots 210 in the corners of the protein array 200. However, the control spots 210 may be positioned at any known locations within the protein array 200 such that registration of other spots 204 relative to the control spots 210 can be performed. Moreover, a different number of control spots 210 may be used in the protein array 200. As another non-limiting example, the control spots 210 may be positioned to minimize the distance between other spots 204 relative to a nearest control spot 210.

The control spots 210 may also be used to indicate if the antibody profile test is working correctly when samples are analyzed. As a non-limiting example, the control spots 210 may be printed with human Immunoglobulin G (IgG) onto the protein array 200. A detection agent may be used to bind with the human IgG of the control spots 210 to form the control complexes. As a result, after completion of the AbP process, if these control spots 210 show a signal, regardless of which individual the sample is from, the identifying steps using the detection agent for the test were done correctly and the test results may be considered valid.

Volume assessment spots 220 also may be included in the embodiment of FIG. 2. Contacting the biological sample with the volume assessment spots 220 the protein array forms volume complexes. Each volume assessment spot 220 may include a predetermined concentration of one or more volume determination proteins. It may be desirable to verify that enough of the biological sample was present in the AbP test to give an accurate result. The volume available from a biological sample can have a huge range. If enough of the biological sample is not utilized, the AbP test may give an invalid result. Volume assessment spots 220 including the volume determination proteins may be used to indicate that the biological sample has sufficient volume to give an accurate result. For this purpose, the volume determination proteins may include two types of protein printed onto the support 202, such as, for example, donkey anti-human Immunoglobulin G and protein G. Both of these proteins will bind human IgG antibodies. The two proteins may be titered with a concentration that will produce a signal when there is enough of the biological sample present.

In one embodiment, a detecting agent binds to the Fc portion of the IgG antibodies is used. And in another embodiment, a detecting agent binds to the FAB portion of the IgG antibodies is used. Or alternatively, a detecting agent binds to the Fc portion of the IgG and a detecting agent binds to the FAB portion of the IgG antibodies are used.

For example, in analysis to determine a suitable concentration, an analysis support may include many different concentrations of the volume determination proteins. Then, different amounts of serum may be contacted with the volume determination proteins. Analysis can determine which concentrations would be suitable to indicate that a minimum amount of serum has been used to produce accurate results for an AbP test. This determined concentration for the volume assessment spots 220 may then be used on a protein array 200 and will indicate with a detectable signal if a sufficient volume of sample has been used in an AbP test.

The location of the volume assessment spots 220 in the protein array 200 of FIG. 2 are examples of one embodiment. Many different locations and number of volume assessment spots 220 may be used.

For the general spots 204 (i.e. not the control spots 210 or volume assessment spots 220), the amount of protein printed for each of the spots 204 may be determined empirically and varied for each spot 204. Some proteins may give a much stronger signal than others may. As a result, the spots 204 may be titered to a lower concentration relative to an average concentration to allow a response that is not saturated. Conversely, low response proteins may be printed at higher concentrations relative to an average concentration to give signals for these proteins that are above a background and improve signal-to-noise ratio.

The size of protein spots 204 on the protein array 200 may be significant for the optimal function of the AbP test. Large spots 204 (e.g., about 600 microns) may give a higher signal and better statistical analysis, but may also have a larger variation in size from print run to print run and within a print run. This larger variation may create inconsistencies between AbP tests and within the same AbP test. Small spots 204 (e.g., about 270 microns) may be more consistent between and within print runs, but often have signals that are too close to a background signal to produce accurate results. Some embodiments may use a spot size of about 340 microns as a balance between sufficient signal-to-noise ratio and sufficient repeatability between print runs.

The trend in the microarray community is to use smaller and smaller spots so that more proteins may be printed per slide. However, with AbP technology a relatively large spot size may produce more accurate and consistent results. With smaller spots sizes, it may be necessary to utilize fluorescent or luminescent detection, which may necessitate the use of expensive scanning systems for data analysis. Forensic laboratories are historically underfunded and may not be able to afford this type of equipment. Thus, for AbP tests it may be more cost effective to use a detection system based on color that can be captured by off the shelf desktop scanners that are readily available to forensic laboratories. Scanning for visible light colors on the protein array 200 may produce more accurate and consistent results with relatively larger spots 204 for use with commercial scanners with sufficient resolution to capture the signals of the larger spots 204.

Moreover, using color produces a more persistent (i.e., non-transient) result that will remain stable for a long time period relative to fluorescent or luminescent type detection systems. As a result, a protein array 200 using visible light colors may be rescanned at some future time if necessary. Fluorescent and luminescent signals are transient and are lost if not scanned within a short time window.

In some AbP processes, the rinsing protocols originally developed for a strip format may not produce acceptable results for a microarray format and may result in high levels of background signal. For example, during some acts in the process fluid may become trapped underneath the glass of a microarray slide and may not be washed away adequately. This trapped fluid may result in high background levels during analysis.

For some embodiments, the slides may be removed from the tray after certain steps (e.g., the blood incubation step and the antibody detection step). With the slides removed, the trays may be quickly rinsed with a buffer to remove trapped liquid and then the slides may be returned to the trays. This change in protocol substantially eliminates the background signal levels due to trapped fluid.

FIG. 3 shows a super array 300 including three protein arrays 310, 320, and 330 according to one or more embodiments of the present disclosure. As an alternate description, the super array 300 may be referred to as a protein array 300 and the protein arrays 310, 320, and 330 may be referred to as sub-arrays. The forensic science community may place significant requirements that results from a given test be statistically valid. Including multiple protein arrays 310, 320, 330 addresses the statistical validity issue by having three tests performed at the same time that should produce near identical (at least with statistical terms) results. Moreover, the results from each sub-array can then be averaged and utilized to perform various statistical analyses. The number of sub-arrays, and their relative positioning may vary greatly and be adjusted based on the type and accuracy of the statistical analysis desired.

It should be emphasized that the embodiments described herein are merely possible examples of implementations, merely set forth for a clear understanding of the principles of the present disclosure. Many variations and modifications may be made to the described embodiment(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

One should note that conditional language, such as, among others, “can,” “could,” “might,” or “may,” unless specifically stated otherwise, or otherwise understood within the context as used, is generally intended to convey that certain embodiments include, while alternative embodiments do not include, certain features, elements and/or steps. Thus, such conditional language is not generally intended to imply that features, elements and/or steps are in any way required for one or more particular embodiments or that one or more particular embodiments necessarily include logic for deciding, with or without user input or prompting, whether these features, elements and/or steps are included or are to be performed in any particular embodiment. Unless stated otherwise, it should not be assumed that multiple features, embodiments, solutions, or elements address the same or related problems or needs.

Various implementations described in the present disclosure may include additional systems, methods, features, and advantages, which may not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

What is claimed is:

1. A method for identifying a source of a biological material, comprising:
contacting a sample of a biological material having individual-specific antibodies with an array including multiple proteins comprising less than about 200 proteins on a support to bind at least a portion of the individual-specific antibodies to the multiple proteins of the array, to form immune complexes;
applying to the array at least one detection agent comprising at least one interacting protein conjugated to a marker, and contacting the detection agent with a plurality of control spots in the array to form control complexes, wherein each control spot of the plurality includes human Immunoglobulin G;
removing non-immobilized individual-specific antibodies and unbound detection agent;
detecting the immune complexes on the array for obtaining an antibody profile; and
detecting an intensity of the control complexes to determine if results of the identifying are complete.

2. The method of claim 1, further comprising comparing the antibody profile to a known antibody profile obtained from an individual.

3. The method of claim 1, further comprising correlating a position of at least some of the plurality of control spots relative to other spots of the array including the multiple proteins to determine an image registration for the detecting the immune complexes.

4. The method of claim 1, wherein the detecting agent binds to the Fc portion of the IgG.

5. The method of claim 1, wherein the detecting agent binds to the FAB portion of the IgG.

6. The method of claim 1, wherein two detecting agents are used, one binding to the Fc portion of the IgG, and the other binding to the FAB portion of the IgG.

7. The method of claim 1, further comprising:
contacting the sample with a plurality of volume assessment spots in analysis the array to form volume complexes, each volume assessment spot including a predetermined concentration of one or more volume determination proteins; and
detecting an intensity of the volume complexes to determine if a volume of the sample is sufficient for an accurate result;
wherein applying to the array the at least one detection agent comprising the at least one interacting protein conjugated to the marker is further performed to detect the volume complexes.

8. The method of claim 3, wherein the one or more volume determination proteins include protein G or donkey anti-human Immunoglobulin G.

9. The method of claim 1, further comprising titrating at least some of the multiple proteins known to develop stronger reactions onto their respective spots of the array in a lower concentration than an average titration.

10. The method of claim 1, further comprising titrating at least some of the multiple proteins known to develop weaker reactions onto their respective spots of the array in a higher concentration than an average titration.

11. The method of claim 1, wherein applying at least one detection agent to the array comprises applying a detection agent comprising at least one interacting protein conjugated to a color marker and to an array with spot sizes sufficiently large as to generate a reaction that produces consistent intensity for high signal-to-noise ratio detection of the color marker on scanning equipment.

12. The method of claim 1, further comprising, after the immune complexes are formed, removing the support from a processing tray, rinsing the processing tray with a buffer, and returning the support to the processing tray for subsequent processing.

13. The method of claim 1, further comprising, after the act of detecting the immune complexes, removing the support from a processing tray, rinsing the processing tray with a buffer, and returning the support to the processing tray for subsequent processing.

14. The method of claim 1, wherein the acts of contacting the sample with the array, applying to the array the at least one detection agent, removing the non-immobilized individual-specific antibodies and the unbound detection agent, detecting the immune complexes on the array, detecting an intensity of the control complexes, and comparing the antibody profile to the known antibody profile are performed on a super array including three or more instances of the array including the multiple proteins, wherein each instance of the super array includes the same multiple proteins in known locations.

15. The method of claim 1, further comprising obtaining the sample of the biological material, and wherein obtaining the sample comprises obtaining a sample of a biological material selected from the group of biological material consisting of tissue, blood, saliva, urine, perspiration, tears, semen, serum, plasma, amniotic fluid, pleural fluid, cerebrospinal fluid, and combinations thereof.

16. A method for identifying a source of a biological material, comprising:
contacting a sample of a biological material having individual-specific antibodies with an array including multiple proteins comprising less than about 200 proteins on a support to bind at least a portion of the individual-specific antibodies to the multiple proteins of the array, to form immune complexes;
contacting the sample with a plurality of volume assessment spots in the array to form volume complexes, each volume assessment spot including a predetermined concentration of one or more volume determination proteins;
applying to the array at least one detection agent comprising at least one interacting protein conjugated to a marker to detect the immune complexes and the volume complexes;
removing non-immobilized individual-specific antibodies and unbound detection agent;
detecting the immune complexes on the array to obtain an antibody profile;
detecting an intensity of the volume complexes to determine if a volume of the sample is sufficient for an accurate result.

17. The method of claim 16, further comprising comparing the antibody profile to a known antibody profile obtained from an individual.

18. The method of claim 16, wherein the detecting agent binds to the Fc portion of the IgG.

19. The method of claim 16, wherein the detecting agent binds to the FAB portion of the IgG.

20. The method of claim 16, wherein two detecting agents are used, one binding to the Fc portion of the IgG, and the other binding to the FAB portion of the IgG.

21. The method of claim 16, wherein the one or more volume determination proteins include protein G or donkey anti-human Immunoglobulin G.

22. The method of claim 16, wherein applying to the array at least one detection agent further includes:
contacting the at least one detection agent with a plurality of control spots in the array to form control complexes, wherein each control spot of the plurality includes human Immunoglobulin G; and
detecting an intensity of the control complexes to determine if results of the identifying are complete.

23. The method of claim 22, further comprising correlating a position of at least some of the plurality of control spots relative to other spots of the array including the multiple proteins to determine an image registration for the detecting the immune complexes.

24. The method of claim 16, further comprising titrating at least some of the multiple proteins known to develop stronger reactions onto their respective spots of the array in a lower concentration than an average titration.

25. The method of claim 16, further comprising titrating at least some of the multiple proteins known to develop weaker reactions onto their respective spots of the array in a higher concentration than an average titration.

26. The method of claim 16, wherein applying at least one detection agent to the array comprises applying a detection agent comprising at least one interacting protein conjugated to a color marker and to an array with spot sizes sufficiently large as to generate a reaction that produces consistent intensity for high signal-to-noise ratio detection of the color marker on scanning equipment.

27. The method of claim 16, further comprising, after the immune complexes are formed, removing the support from a processing tray, rinsing the processing tray with a buffer, and returning the support to the processing tray for subsequent processing.

28. The method of claim 16, further comprising, after the act of detecting the immune complexes, removing the support from a processing tray, rinsing the processing tray with a buffer, and returning the support to the processing tray for subsequent processing.

29. The method of claim 16, wherein the acts of contacting the sample with the array, contacting the sample with the plurality of volume assessment spots, applying to the array the at least one detection agent, removing the non-immobilized individual-specific antibodies and the unbound detection agent, detecting the immune complexes on the array, detecting an intensity of the volume complexes, and comparing the antibody profile to the known antibody profile are performed on a super array including three or more instances of the array including the multiple proteins, wherein each instance of the super array includes the same multiple proteins in known locations.

30. The method of claim 16, further comprising obtaining the sample of the biological material, wherein obtaining the sample comprises obtaining a sample of a biological material selected from the group of biological material consisting of tissue, blood, saliva, urine, perspiration, tears, semen, serum, plasma, amniotic fluid, pleural fluid, cerebrospinal fluid, and combinations thereof.

31. A protein array, for identifying an individual, comprising:
an array of multiple proteins comprising less than about 200 proteins immobilized on a support, wherein each protein is known, each protein is immobilized at a known predetermined location on the support, and the multiple proteins are configured to bind to at least a portion of individual-specific antibodies to form immune complexes; and
a plurality of control spots as part of the array, wherein each control spot includes human Immunoglobulin G configured to form control complexes.

32. The protein array of claim 31, further comprising a plurality of volume assessment spots as part of the array, wherein each volume assessment spot includes a predetermined concentration of one or more volume determination proteins configured to bind to antibodies of a human to form volume complexes.

33. The protein array of claim 31, wherein the one or more volume determination proteins include protein G, donkey anti-human Immunoglobulin G, or a combination thereof.

34. The protein array of claim 31, wherein at least some of the multiple proteins known to develop stronger reactions are titrated onto their respective spots of the array in a lower concentration than an average titration.

35. The protein array of claim 31, wherein at least some of the multiple proteins known to develop weaker reactions are titrated onto their respective spots of the array in a higher concentration than an average titration.

36. The protein array of claim 31, wherein each of the multiple proteins is configured to conjugate to a color marker when a detection agent is applied thereto and a spot size of each of the multiple proteins is sufficiently large as to generate a reaction that produces consistent intensity for high signal-to-noise ratio detection of the color marker on scanning equipment.

37. The protein array of claim 36, wherein the spot size is about 340 microns.

38. A protein array, for identifying an individual comprising:
an array of multiple proteins comprising less than about 200 proteins immobilized on a support, wherein each protein is known, each protein is immobilized at a known predetermined location on the support, and the multiple proteins are configured to bind to at least a portion of individual-specific antibodies to form immune complexes; and
a plurality of volume assessment spots as part of the array, wherein each volume assessment spot includes a predetermined concentration of one or more volume determination proteins configured to bind to antibodies of a human to form volume complexes.

39. The protein array of claim 38, wherein the one or more volume determination proteins include protein G, donkey anti-human Immunoglobulin G, or a combination thereof.

40. The protein array of claim 38, further comprising a plurality of control spots as part of the array, wherein each control spot includes human Immunoglobulin G configured to form control complexes.

41. The protein array of claim 38, wherein at least some of the multiple proteins known to develop stronger reactions are titrated onto their respective spots of the array in a lower concentration than an average titration.

42. The protein array of claim 38, wherein at least some of the multiple proteins known to develop weaker reactions are titrated onto their respective spots of the array in a higher concentration than an average titration.

43. The protein array of claim 38, wherein each of the multiple proteins is configured to conjugate to a color marker when a detection agent is applied thereto and a spot size of each of the multiple proteins is sufficiently large as to generate a reaction that produces consistent intensity for high signal-to-noise ratio detection of the color marker on scanning equipment.

44. The protein array of claim 43, wherein the spot size is about 340 microns.

45. A protein array, for identifying an individual, comprising:
a plurality of sub-arrays, each sub-array comprising:
an array of multiple proteins comprising less than about 200 proteins immobilized on a support, wherein each protein is known, each protein is immobilized at a known predetermined location on the support, and the multiple proteins are configured to bind to at least a portion of individual-specific antibodies to form immune complexes; and
a plurality of control spots as part of the array, wherein each control spot includes human Immunoglobulin G configured to form control complexes.

46. A protein array, for identifying an individual comprising:
a plurality of sub-arrays, each sub-array comprising:
an array of multiple proteins comprising less than about 200 proteins immobilized on a support, wherein each protein is known, each protein is immobilized at a known predetermined location on the support, and the multiple proteins are configured to bind to at least a portion of individual-specific antibodies to form immune complexes; and
a plurality of volume assessment spots as part of the array, wherein each volume assessment spot includes a predetermined concentration of one or more volume determination proteins configured to bind to antibodies of a human to form volume complexes.
